(19) United States
(12) Patent Application Publication
(10) Pub. No.: US 2017/0112961 A1
(43) Pub. Date: Apr. 27, 2017

(54) PRODUCTS FOR REPAIRING CARTILAGE LESIONS, METHOD OF PREPARATION AND USES THEREOF

(71) Applicant: STEMMATTERS, BIOTECNOLOGIA E MEDICINA REGENERATIVA SA, Barco GMR (PT)

(72) Inventors: Rui Pedro Romero Amandi de SOUSA, Barco GMR (PT); Cristina CORREIA, Barco GMR (PT); Carlos Alberto VILELA GOMES, Barco GMR (PT); Alain José DA SILVA MORAIS, Barco GMR (PT); Ana Catarina FREIRE GERTRUDES, Barco GMR (PT); Tírcia Susete XAVIER CARLOS DOS SANTOS, Barco GMR (PT); Joaquim Miguel ANTUNES CORREIA DE OLIVEIRA, Barco GMR (PT); João Duarte DUARTE COELHO DO SAMEIRO ESPREGUEIRA MENDES, Barco GMR (PT); Rui Luís GONÇALVES DOS REIS, Barco GMR (PT)

(21) Appl. No.: 15/311,796

(22) PCT Filed: May 15, 2015
(86) PCT No.: PCT/IB2015/053604

§ 371 Date: Nov. 16, 2016

(30) Foreign Application Priority Data
May 16, 2014 (PT) 107642

(51) Int. Cl. A61L 27/20; A61L 27/26; A61L 27/38; C08B 37/00; A61L 27/52

(52) U.S. Cl. CPC A61L 27/20; C08B 37/006; A61L 27/52; A61L 27/3852; A61L 27/26; A61L 2430/06; A61L 2400/06

(57) ABSTRACT

Products for repairing cartilage lesions, method of preparation and uses thereof The present disclosure provides products and methods of preparation thereof, said products comprising a matrix of methacrylated gellan gum having a methacrylation degree between 1.5 and 6%, cartilage forming cells and a physiologically acceptable ionic solution containing cations, for application in tissue engineering and regenerative medicine, in particular cartilage lesions.

### TECHNICAL FIELD

The present disclosure relates to products and preparation methods for the treatment of tissues, in particular cartilage lesions, by means of tissue engineering and regenerative medicine. A composition includes a matrix and cartilage forming cells.

### BACKGROUND ART

Articular cartilage tissue is composed by one single cell type—chondrocytes, a dense extracellular matrix which constitutes 20% of the tissue, while the remaining composition of cartilage (approximately 80%) is water. It completely lacks nervous and vascular systems, which are those mostly involved in tissue repair mechanisms. Cartilage tissue is well known by those skilled in the art to have very limited repair capabilities when injured.

The concomitant increase in life expectancy worldwide and physical activity throughout life time, in which sport activities play an important role as they are practiced up to seniority, increases risk for traumatic lesions of the cartilage tissue, as well as increases likelihood of cartilage wear by use. If not treated in a short period of time, cartilage lesions will ultimately evolve to a degenerative state, where the only remaining option is total replacement of the joint by prosthesis. As a consequence, cartilage lesions can lead to physical impairment and interruption of normal daily activities, causing absence from work and/or school and premature abandonment of sports activity. First line of treatment involves long-term physical therapy and drugs, which render limited efficacy. Cartilage lesions have high direct and indirect impact, both in terms of quality of life and economic dimensions.

The demand for treatments with durable outcomes is of utmost interest not only for patients but also for physicians, who need a durable solution to offer to their patients. Currently, there is yet no fully clinically acceptable therapeutic option for focal cartilage lesions.

Tissue transplantation procedures such as periosteum, perichondrium, and osteochondral grafts have yielded positive short-term results, but the long-term clinical results are doubtful [Benthien, J. P., et al., Knee Surg Sports Traumatol Arthrosc, 2011. 19(4): p. 543-52.]. Furthermore, tissue availability for transplant constitutes a major limitation, especially in large cartilage defects.

The above limitations have motivated the development of cell based approaches for treatment of cartilage defects. The autologous chondrocyte implantation (ACI) procedure has been the cell-based therapy most widely used [Brittberg, M., et al., N Engl J Med, 1994. 331(14): p. 889-95]. However, ACI application may be inadequate in certain scenarios because of anatomic factors, and difficulty of fixation, in degenerative defects, of the periosteal flap or collagen sheets to retain the chondrocyte suspension. Other potential complications include periosteal hypertrophy, ablation, uneven cell distribution, and loss of cells into the joint cavity resulting in the need of repetition of surgery in up to one third of the patients. Autologous Chondrocyte Transplantation (ACT) represents the only clinical mass available cell-based therapy for cartilage repair. However, even this therapy presents several performance drawbacks resulting in surgical complications, which normally leads to repetition of surgery in 25 to 36% of the ACT treated patients [Harris, J. D., et al., Osteoarthr Cartilage, 2011. 19(7): p. 779-91].

Improvements have aimed to overcome the intrinsic technical disadvantages of first generation ACI by using cartilage tissue engineering (TE) grafts developed with three-dimensional matrices that contain autologous chondrocytes (MACI—matrix-induced autologous chondrocyte implantation) for cartilage regeneration [Brun, P., et al., J Biomed Mater Res, 1999. 46(3): p. 337-46]. Biomaterials that have been used include hyaluronic acid and collagen type I and III. Several alternative TE approaches have been investigated in an effort to engineer cartilage in vitro to produce grafts that will facilitate regeneration of articular cartilage. In this approach, chondrocytes are seeded into various biocompatible scaffolds and either further cultured under chondrogenic conditions or implanted immediately leading to the second and third generation ACI. These new approaches still require improvements both at material, cellular and surgical method dimensions.

The growing clinical demand, as well as the increasing industrial interest, of therapeutic approaches for cartilage repair has been reflected by the increase in scientific literature and intellectual property landscapes regarding methods and compositions intended to promote cartilage regeneration. These methods and compositions may include exclusively materials, growth factors, cells or a combination of thereof.

Document US2011/0184381 A1 describes the use of layers of synovium or peritendineum, which contain chondro- and osteo-progenitor cells. These layers are further interposed with layers of a matrix containing chondrogenic factors and anti-hypertrophic agents at the cartilage area, and osteogenic factors at the bone area.

Document US2013/0281378 A1 describes the use of a composite of an electrospun fiber and a hydrogel composed of gelatin and sodium cellulose sulphate.

Document US2013/0287753 A1 describes a composition that includes a platelet-based material, and one or more chondrogenesis inducing agents. Both components can be autologous, used with or without a cell-based therapy.

Document U.S. Pat. No. 6,129,761 A, describes the use of a cell-hydrogel suspension, which is comprised of a biocompatible polymer capable of crosslinking to form a hydrogel containing dispersed cells.

Document US2004258731 A1, describes the use of a formulation comprised by a drug, with a chondrogenesis-promoting action, a biodegradable and/or biocorrosive polymer, and a porous matrix and/or a hydrogel that does not inhibit cartilage repair.

Document US2011/274742 A1 addresses the use of a hydrogel or scaffold compositions, comprised of a water soluble cellulose compound and a fibrous or filamentous matrix, which promotes, facilitates, and/or enhances progenitor or stem cell growth and/or differentiation.

Document US2010120149 A1 addresses the use of a cell aggregate-hydrogel-polymer scaffold, where cell aggregates are of differentiated chondrocytes, dispersed through the hydrogel, and further used to fill the pores of the scaffold.

Document Coutinho, D. F., et al., Biomaterials, 2011, 31, 7494-7502 discloses a modified gellan gum hydrogel with tunable physical and mechanical properties, which is proposed as suitable for a wide range of tissue engineering approaches. In this approach, gellan gum is methacrylated at the hydroxyl group of one of the glucose residues of the tetrasaccharide repeat unit by reaction with methacrylic anhydride to give the methacrylated gellan gum having the structure shown in FIG. 1.

Gellan gum is a linear, anionic heteropolysaccharide consisting of a glucose-glucuronic acid-glucose-rhamnose tetrasaccharide repeating unit. It is commercially available in two forms, acetylated and deacetylated, known as high and low-acyl forms, respectively. In the high acyl form, gellan gum has one glycerate group and 0.5 acetate substituents per tetrasaccharide repeating unit and these acyl substituents are located on the same glucose residue. In the low acyl form, gellan gum contains no acyl substituents. Gellan gum contains several hydroxyl substituents, as well as one free carboxylic group per repeating unit which can be used for further functionalisation of the polymer.

Through modification of the reaction conditions, the methacrylated gellan gum was obtained with two degrees of substitution (fraction of modified hydroxyl groups per repeating unit), namely 1.2% and 11.25%, as determined from analysis of the ¹H NMR spectrum. Both materials were shown capable of hydrogel formation through photo- and ionic-crosslinking mechanisms (UV light and cations such as Ca²⁺, respectively). However, it is noted that ionic-crosslinked hydrogels made from methacrylated gellan gum with high substitution degree have poor mechanical properties. Ideally, the methacrylated gellan gum should form stable hydrogels via either crosslinking method, preferably via ionic-crosslinking.

Furthermore, the dissolution of both high and low substituted methacrylated gellan gum in water is reported to take place at 50° C. for solutions with concentrations between 0.5 and 2% w/V. This aspect represents a limitation in terms of the need for additional equipment and time required for obtaining homogeneous solutions of the materials. Furthermore, this temperature is incompatible for applications in animals and humans, wherein physiological temperature is approximately 37° C. Ideally, the methacrylated gellan gum should be readily soluble in water between room temperature and 37° C.

Finally, the in vitro cell viability of photoencapsulated NIH-3T3 cells was assessed via a standard live/dead assay in hydrogels made from methacrylated gellan gum with high (11.25%) and low (1.2%) substitution degrees. After 3 days, the fluorescence micrograph images shown in FIG. 7 of this publication show few live cells (stained green) in hydrogels made from methacrylated gellan gum with low substitution degree. Conversely, close analysis of the image of cells encapsulated in hydrogels made from methacrylated gellan gum with high substitution degree show a very significant number of red stains, which is indicative of dead cells.

Document WO2011/119059 A1 discloses an alternatively modified, photo-crosslinkable gellan gum, which is proposed for tissue engineering and regenerative medicine applications. In this approach, gellan gum is methacrylated at the carboxyl group of the glucuronic acid residue of the tetrasaccharide repeat unit by reaction with glycidyl methacrylate to give the methacrylated gellan gum having the structure shown in FIG. 2 of the present disclosure.

According to the 1H NMR spectrum presented in FIG. 5 of WO2011/119059 A1, the degree of substitution is low, approximately 0.7%. This material is described as readily soluble in water at 37° C. at a concentration of 2% w/V. After dissolution in water, a photo-initiator such as methyl benzoylformate (MBF) or hydroxy-4′-(2-hydroxyethoxy)-2-methylpropiophenone (HHMPP) is added and hydrogel formation is promoted by photo-crosslinking by the action of UV light.

While this alternative methacrylated gellan gum having very low substitution (<1%) degree may be perceived as having some advantages, several shortcomings may be identified in the specification by the skilled artisan. While photo-crosslinking promotes hydrogel formation, this reaction requires catalysis by a photo-initiator, the majority of which are known to be cytotoxic even at very low concentrations, provoking cell death. Free radicals formed during the photoreaction also have negative impact on cell viability. Finally, the photo-crosslinked gels are further equilibrated by contact with a liquid, such as phosphate buffered saline (PBS). Ideally, methacrylated gellan gum should form stable hydrogels through ionic-crosslinking in a single step, without the need for additional cell-toxic reagents, UV light or further processing steps.

Despite the evolution in cartilage treatments, most methods only result in temporary improvement of clinical symptoms, such as pain relief, while the regeneration of long-lasting hyaline cartilage tissue remains a significant challenge. In addition, most of these approaches involve two surgical steps, one for articular cartilage biopsy and another for tissue engineered (TE) product application.

The biopsy procedure of cartilage for subsequent chondrocyte isolation causes site morbidity and increases cost of the overall procedure. In addition, the expansion of chondrocytes to therapeutic relevant numbers is lengthy and prone to in vitro cell dedifferentiation, and chondrocytes are commonly incapable of redifferentiation after implantation, leading to formation of fibrous cartilage tissue. This fact requires chondrocytes to be redifferentiated before implantation, which further contributes to increase treatment cost.

Current cell therapies also fail due to unfavorable microenvironments for cells: on one side, cells require a biomaterial support to be retained in the lesion site, and avoid spreading within the joint cavity or even migration to the blood stream. Many supports do not promote native morphology of cartilage cells, as these are naturally in a round shape and surrounded by water, within a dense matrix.

In terms of treatment efficacy, focal lesions with areas above 2 square centimeters are especially demanding for compositions and method of application. Commonly, the irregular surface of cartilage tissue of the joint leads to so-called “kissing lesions” on the opposing surface of the joint, such as the medial tibial plateau. Large defects areas pose additional difficulties, as they demand high cell numbers which increase cell expansion requirements and demand a full open joint procedure.

The fixation of the periosteal flap or biomaterial sheet to the defect border is also technically demanding and, in the case of TE products, the adaptation of the construct to the defect geometry involves cutting and stacking the construct (usually a membrane) according to a mold of the defect, which further increase complexity of the procedure and form an obstacle for the implementation of minimally invasive procedures like arthroscopic related ones. In many cases, the surgery may also involve bone marrow stimulation by drilling of the subchondral bone. The maintenance of a barrier between synovial fluid/cavity and subchondral bone is important as lipids from bone marrow and subchondral bone can enter the joint and intra-articular lipid droplet phagocytosis may be a stimulus for inflammatory arthritis; while on the opposite direction, growth factors present in the synovial fluid may promote subchondral bone overgrowth within the lesion site, which can result in undesirable ossification and ultimately to cartilage thinning at the defect site.

These facts are disclosed in order to illustrate the technical problem addressed by the present disclosure.

### GENERAL DESCRIPTION

Despite many efforts, the challenge in achieving long term cartilage repair is yet to be attained. The ideal cartilage repair product should address simultaneously several product and performance requirements. The ideal TE product should bring together the advantages of an extracellular matrix together with regenerative cells, and should be applied by methods that are minimally invasive and minimize morbidity at the joint. The matrix should be biocompatible and should withstand cell seeding and cell culture protocols, including encapsulation protocols able to be implemented previous to or during the surgical procedure. The material should support viability of mammalian cells at high cell densities, both in vitro and in vivo, preferably human progenitor cells, either autologous or allogeneic.

Accordingly, the present disclosure provides a methacrylated gellan gum having degree of methacrylate substitution appropriate to confer improved aqueous solubility at room or physiological temperature (20° C.-37° C.), to form more stable hydrogels and to maintain higher cell viability for longer periods after encapsulation within the hydrogel.

Surprisingly, it has been found that methacrylated gellan gum with a substitution degree between 1.5 and 6%, in particular 1.5 and 5% provides a particularly suitable matrix for encapsulation of such cells at surgical room temperature, facilitating preparation of the TE product. FIG. 3 shows evidence of high cell viability (>90%) when encapsulated in 2% w/V methacrylated gellan gum at room temperature, and where gel formation occurs by ionic crosslinking. Encapsulated cells include those particularly relevant for cartilage repair, including human articular chondrocytes and human adipose stem cells, and viability was assessed for long time-periods (such as 3 weeks), given the high relevance of this parameter for the purpose of this invention.

Human cells (chondrocytes and adipose stem cells) were encapsulated at room temperature at 10 million cells/mL within a 2% w/V methacrylated gellan gum solution, and 3D gels were formed by ionic crosslinking. Cell viability was assessed by Live/Dead assay after one and three weeks of in vitro culture. Live cells are stained green (whole cytoplasm), while dead cells are detected by red staining of cell nuclei, evidenced as smaller dots.

In an embodiment, such product may be liquid or viscous, and may be injectable under physiological conditions via a simple arthroscopic procedure. The surgical procedure itself may be single step and should not depend on any prior cartilage biopsy, in order to reduce site morbidity and reduce surgery time, as well as to reduce total cost. Upon application of the TE product of the present disclosure, the defect should be easily filled by the product and should be fixed without the use of a periosteal flap. In addition, subchondral drilling should be preferably avoided in the case of chondral lesions (defect of cartilage alone without damage of the underlying bone).

In an embodiment, in a short period of time, the biomaterial should become more rigid, allowing for cell retention at the defect site, acting as a barrier between subchondral bone and the synovial fluid and allowing for an extracellular environment that promotes chondrogenesis of the cells and speeds up cartilage regeneration.

In this regard, the disclosed subject matter addresses the treatment of focal cartilage defects in particular joints by describing a composition and method of use that synergistically address the limitations of current available methods and improves the final outcomes as compared to current standard of care.

The present disclosure also relates to methods and compositions for the treatment of cartilage lesions in animals, particularly in humans by a tissue engineered product combining a matrix and cells and applied by a surgical procedure, preferably by a minimally invasive procedure. The methods and compositions disclosed in this invention promote regeneration of focal cartilage lesions, both superficial and full-thickness.

Another embodiment of the present invention relates to a matrix, in the form of a biodegradable hydrogel, which is delivered to the site of lesion to:
i) reestablish continuity of the cartilage tissue—the matrix functions initially as filler, to restore and preserve function, alleviating pain and minimize progression to osteoarthritis. Said matrix has an adequate mechanical properties, in terms of stiffness and elasticity, in order to respond to the mechanical demands of the joint. Furthermore, the matrix functions as a barrier, avoiding contact between synovial fluid/cavity and subchondral bone.
ii) support an appropriate 3D environment for cell survival and differentiation—The matrix accommodates cells in high density and promotes an environment favoring differentiation along the chondrogenic lineage and development of a hyaline-like cartilage tissue.

Said matrix is composed at least by a methacrylated gellan gum wherein said gellan gum comprises a methacrylation degree between 1.5% and 6%; preferably 1.5% to 5%, even more preferably 3% to 5%, which may optionally contain one or more additives. Additives may include polysaccharides, sulphated polysaccharides, proteins, peptides, and/or growth factors. In an embodiment, the matrix is in a solid or liquid form.

Other embodiment of the present invention relates to cartilage forming cells, such as stem cells, induced pluripotent stem cells and chondrocytes. More preferably, the cartilage forming cells are mesenchymal stromal/stem cells. Alternatively, chondrocytes alone or in combination with stromal/stem cells can be used. These cells are delivered to the site of lesion in combination with said matrix, to:
i) regenerate hyaline cartilage tissue—stromal/stem cells with chondrogenic differentiation potential, such as mesenchymal stromal/stem cells function as a key element in regeneration of hyaline cartilage. Chondrogenic progenitor cells, delivered within said chondrogenic matrix, evolve into the chondrogenic lineage, secrete and deposit extracellular matrix as found in native hyaline cartilage, such as collagen type II and glycosaminoglycans.
ii) avoid cartilage biopsy: The use of stromal/stem cells alone avoids cartilage biopsy and inherent morbidity derived from the harvest procedure, and has advantages in terms of speed and cost during cell expansion, due to their high proliferative capacity. This approach supports the use of autologous or allogeneic cells and enables a single step joint procedure.

An aspect of the disclosed subject matter discloses a methacrylated gellan gum comprising a methacrylation degree between 1.5-6%, preferably with a methacrylation degree between 1.5-5%, more preferably with a methacrylation degree between 3-5%.

In an embodiment of the methacrylated gellan gum of the present subject matter, the gellan gum may have at least one monomeric unit or monomeric subunit having a chemical functional group for binding, in particular wherein the chemical functional group is a carboxylic group.

In an embodiment of the methacrylated gellan gum of the present subject matter, the gellan gum acylation degree may be from no acyl groups up to two acyl substituents, in particular acetate and glycerate, both located on the same glucose residue. In a preferred embodiment, the gellan gum acylation degree is one glycerate per repeat unit and one acetate per every two repeat units. More preferably, the gellan gum has no acyl groups.

In an embodiment of the methacrylated gellan gum of the present subject matter, the methacrylated gellan gum can be used in human or veterinary medicine, preferably for use in regenerative medicine and tissue engineering and more preferably for use in cartilage repair or treatment.

Another aspect of the present disclosure relates to a hydrogel comprising the gellan gum disclosed in the present subject matter.

Another aspect of the present disclosure relates to a composition for use in cartilage tissue engineering and regenerative medicine, comprising a matrix containing methacrylated gellan gum of the present subject matter, mammalian cells and a physiological ionic solution containing cations in an effective amount.

In an embodiment of the composition for use in cartilage tissue engineering and regenerative medicine, comprising a matrix containing methacrylated gellan gum having a methacrylation degree of up to 60%; preferably a methacrylation degree of 1-6%, even more preferably a methacrylation degree of 3-5%; mammalian cells and a physiological ionic solution comprising cations in an effective amount wherein;
the methacrylated gellan gum comprises between 0.5% w/V composition and 4% w/V composition, preferably between 1.5% and 2.5% w/V composition;
the mammalian cells comprise between 0.5 and 60 million cells per mL composition, preferably between 5 and 15 million cells per mL composition;
the physiological ionic solution comprises between 5 and 20% V/V composition, preferably between 8 and 12% V/V composition.

In another embodiment, the matrix of the composition(s) of the present subject may further comprise polysaccharides from the group consisting of hyaluronan, agarose, alginate, chitosan or starch, or mixtures thereof, among others.

In another embodiment, the matrix of the composition(s) of the present subject may further comprise sulphated polysaccharides from the group consisting of chondroitin sulphate, keratan sulphate, heparin sulphate, dermatan sulphate, gellan sulphate or ulvan, or mixtures thereof, among others.

In another embodiment, the matrix of the composition(s) of the present subject may further comprise proteins from the group consisting of collagen type II, collagen type I, fibronectin, gelatin or laminin, or mixtures thereof, among others.

In another embodiment, the matrix of the composition(s) of the present subject may comprises more than 50% V/V methacrylated gellan gum, preferably more than 90% V/V.

In another embodiment, the mammalian cells of the composition(s) of the present subject may be stem cells, in particular from the group consisting of adult mesenchymal stromal/stem cells and/or induced pluripotent stem cells, among others. In a preferred embodiment the mesenchymal stromal/stem cells may be obtained from adipose tissue.

In another embodiment, the mammalian cells of the composition(s) of the present subject may be cartilage forming cells, namely chondrocytes or chondrocytes combined with stem cells, among others.

In another embodiment, the mammalian cells of the composition(s) of the present subject may be from a donor or the patient subject to the cartilage tissue engineering or regenerative medicine.

In another embodiment, the mammalian cells of the composition(s) of the present subject comprises a sub-population of chondrogenic progenitor cells, from the group expressing markers CD106, CD271, CD29, SOX-9, dlk1/FA1, CD44 and CD151, among others.

In another embodiment, the ionic solution of the composition(s) of the present subject may include a cell culture media, phosphate buffer saline, sodium chloride solution, calcium chloride solution or mixtures thereof, among others.

In another embodiment, said composition of the disclosed subject matter may be in an injectable form, and said injectable composition may be crosslinked in situ.

Another aspect of the present invention relates to a patch, a strip, a mesh, a disc, a scaffold or a membrane comprising the composition or methacrylated gellan gum of the disclosed subject matter.

Another aspect of the present invention is related to a kit for cartilage tissue engineering or regenerative medicine comprising part or all components of said composition, namely a matrix containing methacrylated gellan gum of the present disclosure, mammalian cells and a physiological ionic solution comprising cations.

In an embodiment, the kit may further comprise mammalian cells and a physiological ionic solution comprising cations.

Another aspect of the present invention is related to a method for preparing the composition of the present subject matter, comprising a step of dissolving the matrix in deionized water.

In other embodiment, the matrix is dissolved at a temperature between 15 and 40° C., preferably between 18 and 25° C.

In other embodiment, the dissolution of the matrix is such that the dissolved matrix is in liquid state at a temperature between 15 and 40° C., preferably between 18 and 25° C.

The method of use involves the combination of the matrix with cells. In an embodiment, the cells are combined with the matrix prior to its administration. In another embodiment with even better results, the cells are encapsulated within the matrix and administered during a surgical procedure to the defect site. In an embodiment, the surgical procedure is a minimally invasive procedure.

In an embodiment of the present subject matter, a method of use is meant to:
i) adopt an arthroscopic procedure—the surgical procedure is minimally invasive, allowing for the administration of the product through a port of reduced cross section area. Such method avoids open joint procedures, and reduces pain, risk of post-surgical complications, and speeds up recovery times, while reducing cost of surgery in an outpatient setting.
ii) maintain subchondral bone intact—the cells are delivered within a chondrogenic matrix, which will regenerate chondral tissue. This method dispenses the use of subchondral bone as a reservoir of progenitor cells and maintains bone intact, without any interference with bone homeostasis.

Throughout the description and claims the word “comprise” and variations of the word, are not intended to exclude other technical features, additives, components, or steps. Additional objects, advantages and features of the invention will become apparent to those skilled in the art upon examination of the description or may be learned by practice of the invention. The following examples and drawings are provided by way of illustration and are not intended to limit the present disclosure. Furthermore, the present disclosure covers all possible combinations of particular and preferred embodiments described herein.

### BRIEF DESCRIPTION OF THE DRAWINGS

The foregoing summary of the disclosed subject matter, as well as preferred mode of use and further advantages thereof, will be best understood with reference to the following detailed description of embodiments, when read in conjunction with the following figures. It should furthermore be understood that the figures are provided for illustration purposes, and should not be considered as definition of the limits of the present invention.

FIG. 1. Shows the structure of a methacrylated gellan gum product obtained by reaction of gellan gum with methacrylic anhydride.

FIG. 2. Shows the structure of a methacrylated gellan gum product obtained by reaction of gellan gum with glycidyl methacrylate.

FIG. 3. Shows evidence of high cell viability (>90%) when encapsulated in 2% w/V methacrylated gellan gum at room temperature, and where gel formation occurs by ionic crosslinking. Encapsulated cells include those particularly relevant for cartilage repair, namely human articular chondrocytes and human adipose stromal/stem cells, and viability was assessed for long time-periods (such as 3 weeks), given the high relevance of this parameter for the purpose of the disclosed subject matter.

FIG. 4. Illustrates a flowchart of the method for preparing said composition of the disclosed subject matter, for treatment of cartilage lesions as described in the disclosed subject matter.

FIG. 5. Illustrates the gene expression ratio of collagen type II and collagen type I after 21 days of in vitro culture in chondrogenic conditions, normalized to non-cultured.

FIG. 6. Illustrates microscopic imaging (20×) graft sections stained with safranin O and alcian blue along 21 days of culture.

FIG. 7. Illustrates microscopic imaging (5×) of rabbit knee articular cartilage sections, with induced lesion and treatment. Safranin O staining performed after 8 weeks of treatment. Top: cartilage lesion treated with disclosed composition; Middle: cartilage lesion treated with current standard of care approach—microfracture; Bottom: cartilage lesion untreated.

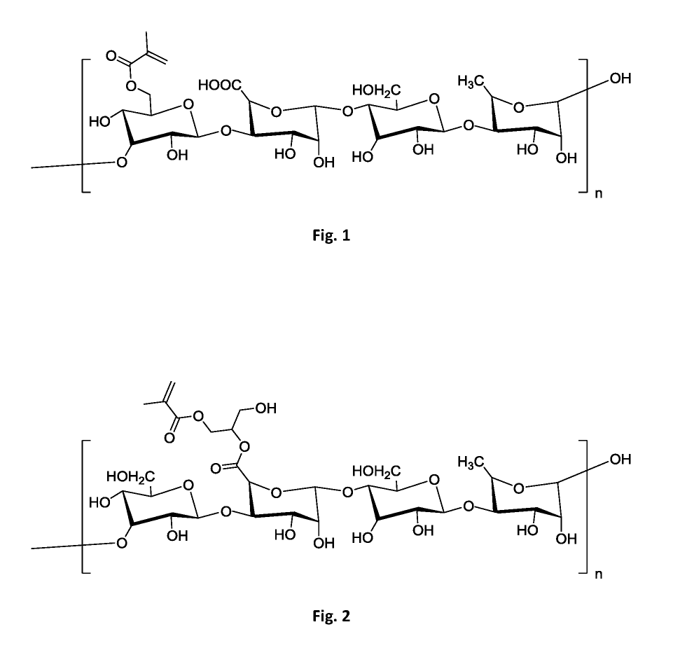

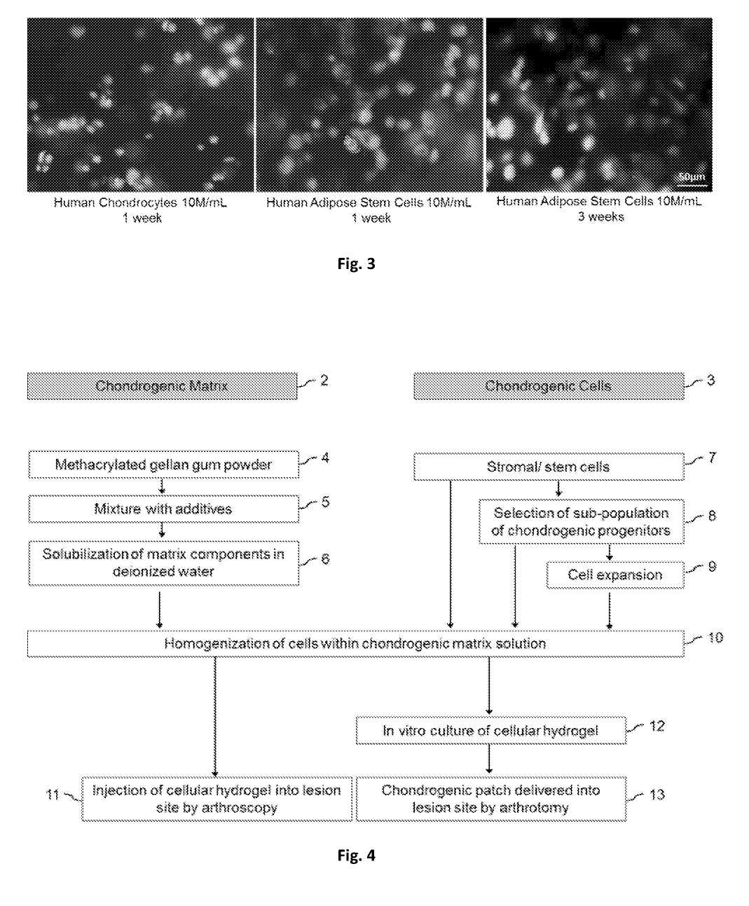

### DETAILED DESCRIPTION

One skilled in the art would understand the following description, as well as terminology used herein, as to best describe the disclosed subject matter, and embodiments chosen to do so are not intended to be exhaustive or to limit the scope to the form disclosed.

In an embodiment, the present disclosure provides a methacrylated gellan gum having a methacrylation degree between 1.5 and 6% appropriate to confer improved aqueous solubility at room and physiological temperature, to form more stable hydrogels and to maintain higher cell viability for longer time after encapsulation of cells within the hydrogel.

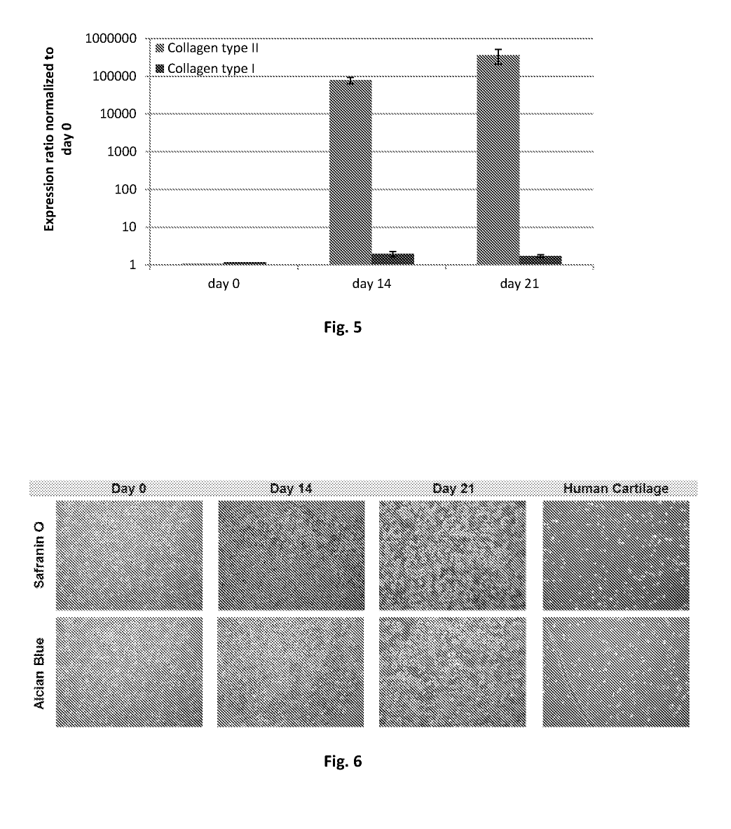

In another embodiment, the present disclosure relates to a composition for treatment of cartilage lesions. Referring to FIG. 4, the composition includes a matrix (2) and cartilage forming cells (3). The matrix is composed totally or partially by polysaccharides, where if more than one polysaccharide is present, these additional polysaccharides are sulphated or non-sulphated. In a preferred embodiment, the main polysaccharide is methacrylated gellan gum (4), with concentrations between 0.5% and 4% w/V, preferably between 1.5 and 2.5% w/V. In another preferred embodiment, other non-sulphated polysaccharides (5) might include hyaluronan, agarose, alginate, or chitosan, at relative amount below 50%, preferably below 10% V/V. In an equally preferred embodiment, sulphated polysaccharides (5) are selected from the group consisting of chondroitin sulphate, keratan sulphate, heparin sulphate, dermatan sulphate, gellan sulphate and/or ulvan, at relative amount below 50%, preferably below 10% V/V. Preferably other non-polysaccharides (5), include proteins such as collagen type II, collagen type I, fibronectin, and/or laminin, at relative amount below 50%, preferably below 10% V/V.

The cells (3) relate to cartilage forming cells. In a preferred embodiment, the cells relate to stromal/stem cells (7), preferably adult mesenchymal stromal/stem cells. In a preferred embodiment, adult mesenchymal stromal/stem cells are obtained from adipose tissue, which can be used immediately after isolation from the patient or sourced alternatively from a Master Cell Bank or from a Working Cell Bank. In this case, the donor of said cells has also been qualified in terms of relevant factors such as age, body mass index, absence of bloodborne pathogens and presence/absence of specific medical conditions. In a preferred embodiment, cells have been qualified for sterility, viability, and expression of mesenchymal stem cell markers. In a more preferred embodiment a sub-population of chondrogenic progenitor cells (8) is selected from the initial stromal/stem cells, such as cells expressing, but not limited to, CD73, CD106, CD271, CD29, SOX-9, dlk1/FA1, CD44 and CD151 markers. In a preferred embodiment, cells are expanded (9) in xeno-free cell culture media to reach the required number of cells, which are used at a passage between 1 and 10, preferably between 3 and 5. In an alternative embodiment, chondrocytes can be used alone or in combination with stromal/stem cells.

In a preferred embodiment, the matrix (2) is dissolved and maintained in deionized water (6), at a temperature between 15 and 40° C., preferably between 18 and 25° C., preferably under mild agitation. Said cells are detached after expansion (9) and counted in order to prepare a cell suspension to be mixed with said chondrogenic matrix. In a preferred embodiment, the number of cells yields a final concentration within the chondrogenic matrix ranging between 0.5 and 100 million cells/mL of matrix suspension (preferably 0.5 and 60 million cells/mL of matrix suspension), preferably between 1 and 30 million cells/mL, preferably between 5 and 15 million cells/mL. In a more preferred embodiment, cells are delivered to the matrix within an ionic solution (10), comprising 5 to 20% V/V of final matrix volume, preferably between 8 and 12% V/V. Also in a preferred embodiment, the ionic solution may include cell culture media, phosphate buffer saline, calcium chloride solution or sodium chloride solution.

In a preferred method of treatment, said mixture of cells and matrix solution is performed at the surgery room, immediately before administration into the focal cartilage lesion. The composition is delivered into the lesion site by injection, by an arthroscopic procedure (11).

Also in a preferred method of treatment, said mixture of cells and matrix solution is used to produce a cellular hydrogel. A chondrogenic patch can be produced using a customized or standard mold. In a preferred embodiment, the mixture of cells and matrix solution is transferred to a designated mold and crosslinked into a solid hydrogel by immersion into said ionic solution. In a preferred embodiment, the mold reproduces the geometry and size of the cartilage lesion in the joint such as the femoral condyle or tibial plate; or alternatively in the hip or ankle joint, among others. In an equally preferred embodiment, a standard chondrogenic patch is produced in a standardized mold with an area below 12 square cm. In a preferred embodiment, the height of the chondrogenic patch is below 3.5 mm, preferably between 2 and 3 mm.

In vitro culture of cellular hydrogel is carried out under chondrogenic conditions (12), including but not limited to, chondrogenic growth factors, and/or dynamic culturing, and/or hypoxic atmosphere. In the said chondrogenic conditions, chondrogenic growth factors include, but are not limited to, transforming growth factor-beta (TGF-β) superfamily such as TGF-β1 and TGF-β3, bone morphogenetic proteins (BMP), including BMP-2, BMP-4, BMP-6 and BMP-7, and growth differentiation factors (GDF), such as GDF-5; but also others such as insulin growth factor (IGF-1) and elements of the fibroblast growth factor family (FGF), including FGF-2 and FGF-18, all at concentration ranging between 1 ng/mL and 100 ng/mL, preferably between 5 and 10 ng/mL. Other chondrogenic supplements include dexamethasone preferably between 0.1 and 0.5 μM; insulin and transferrin, preferably between 5 and 10 μg/mL and selenium preferably between 5 and 10 ng/mL.

In the said chondrogenic conditions, dynamic culturing includes systems such as those applying perfusion of the cell culture media to the chondrogenic patch, and/or hydrostatic pressure, and/or compression, and/or tension, and/or tortion, and/or stretching. In a preferred embodiment, hydrostatic pressure is used ranging between 0.1 and 10 MPa, preferably between 1 and 5 MPa. Furthermore, in the said chondrogenic conditions, hypoxic atmosphere include levels of oxygen within cell culture media below 21%, preferably between 5% and 1%. In a preferred embodiment, in vitro culture of hydrogel patches occurs up to 28 days, preferably between 14 and 21 days.

Said chondrogenic patch is provided to patient point of care, and is further cut into the required shape and size immediately before application into the focal cartilage lesion. The chondrogenic patch is delivered into the cartilage lesion site by press fit, through an arthrotomy procedure (13).

Said chondrogenic patch can also be used as an ex vivo cartilage model to study objects of interest, including, but not limited to, mechanisms of action of bioactive agents, progression of disease and/or effectiveness of pharmacological treatment.

The preferred embodiment comprises a composition and method of treatment that provides an off-the-shelf approach for regeneration of focal cartilage lesions. Such composition and method result in a single step procedure for treatment of said cartilage lesions, which greatly reduces time and costs of surgery operations, greatly reduces risk of joint infection and/or other surgical complications. By use of stromal/stem cells as a component of such composition, joint morbidity is avoided, given that there is no need for harvesting of osteochondral plugs for mosaicplasty or biopsy of cartilage tissue for chondrocyte isolation, to be subsequently used for treatment. Said composition may comprise allogeneic cells, where cells are obtained from independent and qualified cell batches, improving success of tissue regeneration. Ultimately, said composition is subject to strict quality control assays before release, reducing any pre-determined risk of failure. Such allogeneic therapy further allows scalability of manufacturing, becoming more cost-effective compared to current chondrocyte-based products.

Demonstration of the Influence of Gellan Gum Methacrylation Degree for Compatibility with Therapeutic Applications

Methacrylation of gellan gum is a required characteristic for a suitable matrix for application in the simple, successful cell encapsulation process.

Materials and Methods

Gellan gum polysaccharide with different methacrylation degrees (material) were dissolved in sterile deionized water, crosslinked at physiological temperature by ionic force and cells were encapsulated within the hydrogels. Methacrylation degree of the material could be calculated by several methods used in the literature, and can be calculated using equation 1.

$$DS = \frac{\frac{( \int 5.47 peak + \int 6.18 peak )}{2}}{\frac{\int 1.32 peak}{3} \times ( H )} \div ( OH )$$

Equation 1—Equation for calculation of gellan gum methacrylation degree (DS) based on ¹H NMR spectrum (D₂O, 70° C.). Where H: number of protons on the double bond; OH: number of hydroxyls on the gellan gum repeating unit.

Material performance was evaluated for (i) solubility, (ii) crosslinking by ionic force and (iii) viability of encapsulated cells. Material is considered soluble when it is possible to dissolve the material (in lyophilized powder form) using sterile deionized water at room temperature or physiological temperature (37° C.) within 30 minutes (parameter: solubility). Material is considered able to undergo crosslinking by ionic force wherein it is possible to form stable hydrogels at 37° C., by addition of a physiological ionic solution comprising cations (parameter: ionic crosslinking). Material is considered to maintain cells viable when it is possible to identify live cells by incubation of the hydrogel with calcein fluorescent dye after 24 hours of cell culture (parameter: cell viability).

Results

Table I shows that the major difference between tested gellan gum of different methacrylation degrees is the solubility parameter. Gellan gum with a methacrylation degree of 0% is not soluble in sterile deionized water at room temperature or 37° C. in 30 minutes. This material required a dissolution process of 30 minutes in a 90° C. water-bath, resulting in an aqueous solution too hot for physiological applications. This hot solution required a controlled cooling process to 38° C.-40° C., and only then it was possible to encapsulate the cells and to crosslink the material by ionic force. High control of cooling steps is essential to ensure viable cell encapsulation: (i) cooling too fast may induce gelling of the solution without ionic force due to its thermoreversible properties, impeding successful cell encapsulation; (ii) insufficient or inaccurate cooling, with temperatures slightly above 38° C.-40° C. significantly reduce viability of encapsulated cells. For medical applications, a 90° C. heating process followed by a controlled cooling process constitutes significant operational drawbacks due to additional equipment and time requirements. On the other hand, gellan gum with methacrylation degree between 1.5% and 6%, in particular 1.5-5%, is soluble in sterile deionized water at room temperature or 37° C. in 30 minutes, resulting in a homogeneous aqueous solution at physiological temperature. Cells can be immediately encapsulated within the solution and crosslinking occurs by ionic force. No cooling step is required because all steps of the process can be performed at physiological temperature (37° C.). Gellan gum with a methacrylation degree in the range of 1.5-6% surprisingly solves operational problems for cell encapsulation in medical scenarios (physiological temperature).

TABLE I
Evaluation of gellan gum polysaccharide with different
methacrylation degrees in three key parameters: solubility,
ionic reticulation and cell viability.

| | Gellan gum methacrylation degree | |
|---|---|---|
| Parameter | 0% | [1.5%-5%] |
| Solubility (Room Temperature | No | Yes |
| 20° C.) | | |
| Ionic crosslinking | Yes* | Yes |
| Cell viability | Yes* | Yes |

*Results obtained after dissolution of gellan gum 0% methacrylation degree at 90° C. during 30 minutes and followed by controlled cooling to 38° C.-40° C.

### EXAMPLES

The following examples demonstrate qualitative and quantitative data regarding safety and efficacy obtained by the preferred embodiments of the present disclosure, for cartilage repair, and how these outperform current standard of care.

### Example 1—Preparation of Tissue Engineering Product Composition for Cartilage Repair

An aseptic environment was set to prepare the chondrogenic composition. Chondrogenic matrix was prepared by sourcing 20 mg of methacrylated gellan gum powder with a methacrylation degree between 1.5 and 5%. Quality control ensured absence of any microbial contamination, as well as ensuring levels of mycoplasma and endotoxins below limits acceptable for therapeutic use. An aqueous solution was prepared by homogenizing said powder with sterile deionized water, yielding a 2% w/V solution. Homogenization was performed at 37° C. with mild agitation. Chondrogenic cells were prepared by sourcing 10 million human stromal/stem cells, at passage 1-2, from a master cell bank. Said human stromal/stem cells were isolated in xeno-free conditions from adipose tissue of a qualified donor. The donor sample was qualified as for absence of bloodborne pathogens and absence of known medical conditions. Cells were qualified for presence of at least 90% concomitant expression of CD90, CD73 and CD105, as well as less than 2% concomitant expression of CD31, CD34 and CD45. Quality control ensured absence of any microbial contamination, as well as ensuring levels of mycoplasma and endotoxins below limits acceptable for therapeutic use. Said cells were suspended in phosphate buffer saline, 10% V/V of final matrix volume, and mixed with such pre-prepared matrix solution. Final cell concentration within the matrix was 10 million cells/mL. At this stage, the cartilage repair composition was ready for injection into cartilage focal lesion by arthroscopic surgical procedure. After filling of lesion site, saline solution can be applied to aid crosslinking of the chondrogenic composition.

### Example 2—In Vitro Development of Hyaline Cartilage by the Use of Disclosed Composition

Healthy hyaline articular cartilage is evaluated by the composition of its extracellular matrix, which includes mainly collagen type II and glycosaminoglycans. When fibrous cartilage is formed, the composition of extracellular matrix shifts, giving rise to molecules such as collagen type I that render less elasticity to the tissue, thereby becoming less capable to withstand mechanical demands of the joint. This procedure may be applied to the evaluation of any composition of this invention.

### Materials and Methods

The cartilage repair composition, as described in example 1, was cultured in vitro for 21 days, exposed to chondrogenic growth factors. In vitro-developed grafts were collected for histological assessment according to standard procedures. Safranin O and alcian blue stainings were performed to detected cartilage glycosaminoglycans. Other grafts were used for quantitative determination collagen type II and collagen type I of gene expression: cells were collected and mRNA isolated for real time polymerase chain reaction (qRT-PCR). Gene expression of cartilage grafts cultured for 21 days was normalized to uncultured grafts at day 0, and presented as normalized expression ratio, according to Livak and Schmittgen (Methods 25, 402-408, 2011). Data is presented as average±SD.

Results

FIG. 5 represents the normalized expression ratio genes coding for collagen type II, and collagen type I proteins, quantified by real time PCR (qRT-PCR). After in vitro culture, the cartilage repair composition developed hyaline-like cartilage tissue as demonstrated by progressive overexpression of collagen type II along time, instead of collagen type I, which would be indicative of unwanted fibrocartilage-like tissue development.

FIG. 6 demonstrate histological sections of cultured grafts stained with safranin O and alcian blue to detect deposition of cartilage extracellular matrix glycosaminoglycans. A significant progression of matrix deposition is observed along 21 days of culture.

The trend indicated by gene expression and matrix staining provides support that the disclosed composition is adequate for repair of hyaline cartilage lesions.

### Example 3—In Vivo Repair of Rabbit Hyaline Cartilage Lesion by the Use of Disclosed Composition

The performance and efficacy of disclosed composition and method for treatment of focal cartilage lesions was assessed in a rabbit model.

### Materials and Methods

A rabbit model was used to test the efficacy of the disclosed composition and method on the repair of cartilage lesions. A focal articular cartilage lesion was induced to the animal's knee by the use of a biopsy punch and curette. Lesions were immediately treated either with the preferred embodiment described in example 1, or adopting a current standard of care surgical method—microfracture. As control, lesions were left untreated. An 8 week repair period was allowed, after which articular cartilage samples were harvested for histological analysis. Safranin O/fast green staining was performed to identify status of lesion repair.

### Results

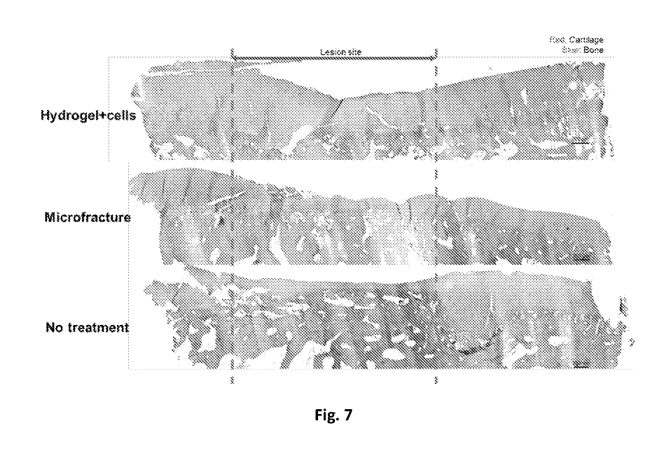

FIG. 7 represents microscopic images of rabbit articular cartilage sections stained with safranin O/fast green, where articular cartilage is stained red, and subchondral bone is stained blue-green. The top image represents staining of lesion treated with preferred composition: 80-90% of cartilage thickness is preserved, integration/bonding with native cartilage occurred, as well as intense and homogenous staining of extracellular matrix throughout the lesion site. The middle image demonstrates staining of lesion treated with microfracture, where the lesion site was mainly filled with bone, and only a thin layer of cartilaginous matrix is observed. This layer is also irregular and bonding with adjacent native cartilage is incomplete. The bottom image represents a lesion that has not been treated: lesion site was also filled with bone due to its overgrowth, and in this case, no cartilaginous matrix was formed, as indicated by the lack of staining by safranin O at the top layer of tissue. This result appears to indicate the formation of fibrous tissue at the surface.

The outcomes of in vivo cartilage repair also provide support that the disclosed composition is adequate for repair of hyaline cartilage lesions, evidence could be seen in FIG. 7.

The disclosure should not be seen in any way restricted to the embodiments described and a person with ordinary skill in the art will foresee many possibilities to modifications thereof.

The above described embodiments are combinable.

The following claims further set out particular embodiments of the disclosure.

1. A methacrylated gellan gum comprising a methacrylation degree between 1.5-6%.

2. The gellan gum according to claim 1, wherein the methacrylation degree is between 1.5-5%.

3. The gellan gum according to claim 1, wherein the methacrylation degree is between 3-5%.

4. The gellan gum according to claim 1, wherein the gellan gum has at least one monomeric unit or monomeric subunit having a chemical functional group for binding, wherein the chemical functional group is a carboxylic group.

5. The gellan gum according to claim 1, wherein the gellan gum acylation degree is from no acyl groups up to two acyl substituents, both located on the same glucose residue with one glycerate per repeat unit and one acetate per every two repeat units.

6. (canceled)

7. The gellan gum according to claim 1 for use in human or veterinary medicine.

8. The gellan gum according to claim 1 for use in regenerative medicine and tissue engineering.

9. (canceled)

10. A hydrogel comprising the gellan gum defined in claim 1.

11. A composition for use in cartilage tissue engineering and regenerative medicine, comprising a matrix containing methacrylated gellan gum as described in claim 1, mammalian cells and a physiological ionic solution comprising cations in an effective amount, wherein the ionic solution includes a cell culture medium, phosphate buffer saline or sodium chloride solution, or mixtures thereof.

12. A composition for use in cartilage tissue engineering and regenerative medicine, comprising a matrix containing methacrylated gellan gum having a methacrylation degree of up to 60%, mammalian cells and a physiological ionic solution comprising cations in an effective amount, wherein
the methacrylated gellan gum is between 0.5% w/V_{composition} and 4% w/V_{composition};
the mammalian cells are between 0.5 and 60 million cells per mL_{composition}; and
the physiological ionic solution is between 5 and 20% V/V_{composition}.

13. The composition according to claim 11, wherein the matrix further comprises at least one of:
polysaccharides from the group consisting of hyaluronan, agarose, alginate, chitosan or starch, or mixtures thereof;
sulphated polysaccharides from the group consisting of chondroitin sulphate, keratan sulphate, herparin sulphate, dermatan sulphate, gellan sulphate or ulvan, or mixtures thereof; and
proteins from the group consisting of collagen type II, collagen type I, fibronectin, gelatin or laminin, or mixtures thereof.

14. (canceled)

15. (canceled)

16. The composition according to claim 11, wherein the matrix comprises more than 50% V/V methacrylated gellan gum.

17. The composition according to claim 11, wherein the mammalian cells are chondrocytes or stem cells from the group consisting of adult mesenchymal stromal/stem cells and/or induced pluripotent stem cells from a donor or the patient subject to the cartilage tissue engineering and regenerative medicine.

18. (canceled)

19. (canceled)

20. (canceled)

21. (canceled)

22. The composition according to claim 11, wherein the mammalian cells comprise a sub-population of chondrogenic progenitor cells, from the group expressing markers CD106, CD271, CD29, SOX-9, dlk1/FA1, CD44 and CD151.

23. (canceled)

24. The composition according to claim 11, wherein said composition is in an injectable form which is crosslinked in situ.

25. (canceled)

26. The composition according to claim 11, wherein the composition is a patch, strip, mesh, disc, scaffold or membrane.

27. A kit for cartilage tissue engineering or regenerative medicine comprising part or all components of the composition according to claim 11, namely a matrix containing methacrylated gellan gum, mammalian cells and a physiological ionic solution comprising cations.

28. (canceled)

29. (canceled)

30. (canceled)

31. (canceled)

32. (canceled)

33. (canceled)

34. The composition of claim 11 for use in, enhancing hyaline cartilage regeneration.
